(19) United States
(12) Patent Application Publication
(10) Pub. No.: US 2024/0324983 A1
(43) Pub. Date: Oct. 3, 2024

(54) AUSCULTATION DEVICE, AUSCULTATION SYSTEM EQUIPPED WITH THE SAME, AUSCULTATION METHOD, AND AUSCULTATION PROGRAM

(71) Applicant: OMRON CORPORATION, Kyoto-shi (JP)

(72) Inventors: Koji MORITA, Kyoto-shi (JP); Tatsuya ADACHI, Kyoto-shi (JP); Hiroyuki ONITSUKA, Kyoto-shi (JP)

(73) Assignee: OMRON CORPORATION, Kyoto-shi (JP)

(21) Appl. No.: 18/443,431

(22) Filed: Feb. 16, 2024

(30) Foreign Application Priority Data
Mar. 30, 2023 (JP) 2023-055657

(51) Int. Cl. A61B 7/02

(52) U.S. Cl. CPC A61B 7/02

(57) ABSTRACT

A PC (20) comprises a communication unit (21) and a calculation and determination unit (28). The communication unit (21) acquires heart sound data from a stethoscope (10). The calculation and determination unit (28) analyzes the waveform including a sound I, a sound II, a sound III, and a sound IV included in one heartbeat of the heart sound acquired by the communication unit (21). The calculation and determination unit (28) determines whether there is any abnormality in the heart sound on the basis of the analysis result for the waveform including the analyzed sound I, sound II, sound III, and sound IV.

### CROSS-REFERENCE TO RELATED APPLICATIONS

THIS APPLICATION CLAIMS PRIORITY TO JAPANESE PATENT APPLICATION NO. 2023-055657 FILED ON Mar. 30, 2023. THE ENTIRE DISCLOSURE OF JAPANESE PATENT APPLICATION NO. 2023-055657 IS HEREBY INCORPORATED HEREIN BY REFERENCE.

### BACKGROUND

### Technical Field

The present invention relates to an auscultation device for measuring heart sound, an auscultation system equipped with this device, an auscultation method, and an auscultation program.

### Description of the Related Art

Systems that measure heart sound and diagnose whether there are any abnormalities in the health of a subject have been used in recent years.

For example, Patent Literature 1 discloses a heart disease diagnostic device, comprising an input signal processing unit that performs predetermined signal processing on inputted electrical signals based on the heart sound of a living body and outputs heart sound data in order to determine the whether there is any heart disease on the basis of heart sound data; and a heart sound analysis unit that subjects heart sound data in a first interval to frequency conversion and calculates the first interval power spectrum, subjects heart sound data in a second interval to frequency conversion and calculates the second interval power spectrum, and diagnoses whether a living body has heart disease on the basis of the difference between the first interval power spectrum and the second interval power spectrum.

### CITATION LIST

### Patent Literature

Patent Literature 1: JP-A 2015-188511 (U.S. Pat. No. 6,244,244)

### SUMMARY

### Problem to be Solved by the Invention

However, the following problem is encountered with the conventional heart disease diagnostic device described above.

That is, with the heart disease diagnostic device disclosed in the above-mentioned publication, heart sound data in a first interval is subjected to frequency conversion and the first interval power spectrum is calculated, heart sound data in a second interval is subjected to frequency conversion and the second interval power spectrum is calculated, and whether a living body has heart disease is diagnosed on the basis of the difference between the first interval power spectrum and the second interval power spectrum.

Accordingly, the power spectrum is calculated by dividing the heart sound data included in one heartbeat into the systolic phase (first interval) and diastolic phase (second interval), which means that it may be difficult to detect abnormalities with high accuracy.

It is an object of the present invention to provide an auscultation device, an auscultation system equipped with this device, an auscultation method, and an auscultation program, with which abnormal heart sounds can be detected more accurately than in the past.

### Means for Solving Problem

The auscultation device according to the first invention is an auscultation device that acquires heart sound data from a stethoscope that measures the heart sound of a subject, and determines whether there is any abnormality, the auscultation device comprising a data acquisition unit, a data analysis unit, and a determination unit. The data acquisition unit acquires the heart sound data from the stethoscope. The data analysis unit analyzes a waveform including a first sound, a second sound, a third sound, and a fourth sound included in one heartbeat of the heart sound acquired by the data acquisition unit. The determination unit determines whether there is any abnormality in the heart sound on the basis of the analysis result for the waveform including the first sound, the second sound, the third sound, and the fourth sound analyzed by the data analysis unit.

Here, examples of the auscultation device include a PC (personal computer), a smartphone, a tablet terminal, or the like that can communicate with a stethoscope that measures the heart sound of a subject.

The stethoscope that measures the heart sound of the subject may be, for example, a device that is used in a state of being attached to the chest of the subject, or a stethoscope, etc., that is used by a physician or the like to measure the heart sound of the subject.

Also, in acquiring heart sound data from a stethoscope, the data may be acquired via wireless communication, or via wired communication, for example.

The first sound, second sound, third sound, and fourth sound are heart sound sounds included in one heartbeat of the heart, and are made by the movement of the subject's heart (ventricle, atrium).

The first sound is, for example, a sound that occurs when a ventricle of the heart contracts. The second sound is, for example, a sound that occurs when a ventricle begins to expand. The third sound is, for example, a sound that occurs when a ventricle finishes expanding. The fourth sound is, for example, a sound that occurs when an atrium of the heart contracts.

Depending on the subject, all of the first, second, third, and fourth sounds may not be detected. For example, only the first and second sounds may be detected, or only the first, second, and third sounds may be detected, or only the first, second, and fourth sounds may be detected.

The determination of whether there is any abnormality in the heart sound by the determination unit is performed, for example, by comparing to the past heart sound waveform data of the subject, or by comparing to the waveform data for the heart sound of a normal healthy person.

Consequently, the waveform including the first, second, third, and fourth sounds is analyzed from the heart sound data included in one heartbeat, and it can be determined whether there is any abnormality in the heart sound according to the difference from the past heart sound data for the subject for each of the sounds, the difference from the heart sound data for a typical healthy person, etc.

As a result, abnormality in the heart sound can be detected more accurately than in the past.

The auscultation device according to the second invention is an auscultation device that measures the heart sound of a subject and determines whether there is any abnormality, the auscultation device comprising a data measurement unit, a data analysis unit, and a determination unit. The data measurement unit measures the heart sound data. The data analysis unit analyzes a waveform including a first sound, a second sound, a third sound, and a fourth sound included in one heatbeat of the heart sound acquired by the data acquisition unit. The determination unit determines whether there is any abnormality in the heart sound on the basis of the analysis result for the waveform including the first sound, the second sound, the third sound, and the fourth sound analyzed by the data analysis unit.

Here, the auscultation device that measures the heart sound of the subject may be, for example, a device that is used in a state of being attached to the chest of the subject, or a stethoscope, etc., that is used by a physician or the like to measure the heart sound of the subject.

The first sound, second sound, third sound, and fourth sound are heart sound sounds included in one heartbeat of the heart, and are made by the movement of the subject's heart (ventricle, atrium).

The first sound is, for example, a sound that occurs when a ventricle of the heart contracts. The second sound is, for example, a sound that occurs when a ventricle begins to expand. The third sound is, for example, a sound that occurs when a ventricle finishes expanding. The fourth sound is, for example, a sound that occurs when an atrium of the heart contracts.

Depending on the subject, all of the first, second, third, and fourth sounds may not be detected. For example, only the first and second sounds may be detected, or only the first, second, and third sounds may be detected, or only the first, second, and fourth sounds may be detected.

The determination of whether there is any abnormality in the heart sound by the determination unit is performed, for example, by comparing to the past heart sound waveform data of the subject, or by comparing to the waveform data for the heart sound of a normal healthy person.

Consequently, the waveform including the first, second, third, and fourth sounds is analyzed from the heart sound data included in one heartbeat, and it can be determined whether there is any abnormality in the heart sound according to the difference from the past heart sound data for the subject for each of the sounds, the difference from the heart sound data for a typical healthy person, etc.

As a result, any abnormality in the heart sound can be detected more accurately than in the past.

The auscultation device according to the third invention is the auscultation device according to the first or second invention, wherein the determination unit determines whether there is any abnormality in the heart sound by using the analysis result by the data analysis unit, the signal strength and duration of at least one of the first sound, the second sound, the third sound, and the fourth sound, the interval between the waveforms, and at least one of the waveform characteristics.

Consequently, whether there is any abnormality in the heart sound can be detected more accurately than in the past by setting as abnormality determination parameters the signal strength, duration, waveform interval, and waveform characteristics (such as frequency characteristics, etc., obtained by FFT (fast Fourier transform) analysis) for each of the sounds (first sound, second sound, third sound, and fourth sound) included in the waveform of the heart sound.

The auscultation device according to the fourth invention is the auscultation device according to the first or second invention, wherein the determination unit determines whether there is any abnormality in the heart sound by comparing the waveform analyzed by the data analysis unit to a waveform of a past heart sound of the subject.

Consequently, whether there is any abnormality in the heart sound can be easily determined on the basis of the change in the subject's heart sound by comparing the past and current heart sound waveforms of the same subject to determine whether there is any abnormality.

The auscultation device according to the fifth invention is the auscultation device according to the fourth invention, wherein the determination unit determines whether there is any abnormality in the heart sound by comparing the waveform analyzed by the data analysis unit to the waveform of the heart sound of a typical healthy person.

Consequently, whether there is any abnormality in the heart sound can be easily determined by comparing the waveform of the subject's heart sound with the waveform of a typical healthy person's heart sound.

The auscultation device according to the sixth invention is the auscultation device according to the fourth invention, wherein the determination unit compares the waveform of the heart sound analyzed by the data analysis unit to a past heart sound waveform, calculates a difference value of a specific parameter, and determines whether there is any abnormality in the heart sound depending on whether the difference value is at or over a specific threshold.

Consequently, it can be determined that there is an abnormality in the heart sound if, for example, the current heart sound waveform is compared to a past heart sound waveform of the same subject and the difference value of a parameter such as waveform signal strength is at or over a specific threshold.

The auscultation device according to the seventh invention is the auscultation device according to the fifth invention, wherein the determination unit determines that the condition of the subject requires follow-up observation in the event that the difference value of a specific parameter calculated by comparing the waveform of the heart sound analyzed by the data analysis unit to a past heart sound waveform is at or over a specific threshold, and the difference value of a specific parameter calculated by comparing to the waveform of the heart sound of a typical healthy person is below a specific threshold.

Consequently, when a determination is made by combining the result of comparing the subject's current heart sound waveform to a past waveform, with the result of comparing to the heart sound waveform of a typical healthy person, if there is a difference when compared to the subject's own past waveform, but the difference is small when compared with the waveform of a typical healthy person, the condition of the subject can be determined to require follow-up observation.

The auscultation device according to the eighth invention is the auscultation device according to the fifth invention, wherein the determination unit determines that the condition of the subject requires follow-up observation in the event that the difference value of a specific parameter calculated by comparing the waveform of the heart sound analyzed by the data analysis unit to a past heart sound waveform is below a specific threshold, and the difference value of a specific parameter calculated by comparing to the waveform of the heart sound of a typical healthy person is at or over a specific threshold.

Consequently, when a determination is made by combining the result of comparing the subject's current heart sound waveform to a past waveform, with the result of comparing to the heart sound waveform of a typical healthy person, if there is a difference when compared to the subject's own past waveform, but the difference is small when compared with the waveform of a typical healthy person, the condition of the subject can be determined to require follow-up observation.

The auscultation device according to the ninth invention is the auscultation device according to the first or second invention, wherein the determination unit determines whether there is any abnormality in the heart sound by comparing the waveform analyzed by the data analysis unit to the waveform of the heart sound of a typical healthy person.

This makes it easy to determine whether there is any abnormality in the subject's heart sound by comparing the waveform of the subject's heart sound with the waveform of a typical healthy person's heart sound.

The auscultation device according to the tenth invention is the auscultation device according to the first or second invention, wherein the first sound is the sound that occurs when a ventricle of the heart contracts, and the second sound is the sound that occurs when a ventricle begins to expand.

Consequently, it can be determined whether there is any abnormality in the heart sound of the subject by using the first sound produced by the contraction of a ventricle of the heart and the second sound produced when a ventricle begins to expand.

The auscultation device according to the eleventh invention is the auscultation device according to the tenth invention, wherein the third sound is the sound that occurs when a ventricle finishes expanding, and the fourth sound is the sound that occurs when an atrium of the heart contracts.

Consequently, it can be determined whether there is any abnormality in the heart sound of the subject by using the third sound that occurs when a ventricle of the heart finishes expanding, and the fourth sound that occurs when an atrium contracts.

The auscultation device according to the twelfth invention is the auscultation device according to the first or second invention, further comprising a display unit that displays the determination result from the determination unit.

Consequently, the subject or medical staff can be apprised of whether there is any abnormality in the heart sound of the subject by displaying on the display unit the result of determining whether there is any abnormality.

The auscultation device according to the thirteenth invention is the auscultation device according to the first or second invention, further comprising a heart sound reproduction unit that reproduces data about the heart sound on the basis of the determination result from the determination unit.

Consequently, if it is determined that there is an abnormality in the heart sound, for example, the patient or medical staff can check a heart sound that was determined to be abnormal, by reproducing the heart sound determined to be abnormal.

The auscultation device according to the fourteenth invention is the auscultation device according to the first or second invention, further comprising a storage unit that stores the heart sound data and the determination result from the determination unit.

Consequently, by storing measured heart sound data and determination results in the storage unit, the subject or medical staff can read out and check changes in the subject's heart sound and past determination results as needed.

The auscultation system according to the fifteenth invention comprises the auscultation device according to the first or second invention, and a remote terminal to which the heart sound data and the determination results are transmitted from the auscultation device.

Consequently, the measured heart sound data and determination results are transmitted to a PC or other such remote terminal installed in a medical facility, allowing information to be shared with a physician at a remote location to afford a medical examination.

The auscultation method according to the sixteenth invention is an auscultation method for measuring the heart sound of a subject and determining whether there is any abnormality, the method comprising a data acquisition step, a data analysis step, and a determination step. In the data acquisition step, a data acquisition unit of an auscultation device acquires data about the heart sound. In the data analysis step, a data analysis unit of the auscultation device analyzes a waveform including a first sound, a second sound, a third sound, and a fourth sound included in the heart sound acquired in the data acquisition step. In the determination step, the determination unit of the auscultation device determines whether there is any abnormality in the heart sound on the basis of the analysis result for the waveform including the first sound, the second sound, the third sound, and the fourth sound analyzed in the data analysis step.

Here, the first sound, second sound, third sound, and fourth sound are beats included in a single heart sound, and are generated by the movement of the subject's heart (ventricle, atrium).

The first sound is, for example, a sound that occurs when a ventricle of the heart contracts. The second sound is, for example, a sound that occurs when a ventricle begins to expand. The third sound is, for example, a sound that occurs when the ventricle finishes expanding. The fourth sound is, for example, a sound that occurs when an atrium of the heart contracts.

Depending on the subject, all of the first, second, third, and fourth sounds may not be detected. For example, only the first and second sounds may be detected, or only the first, second, and third sounds may be detected, or only the first, second, and fourth sounds may be detected.

The determination of whether there is any abnormality in the heart sound by the determination unit is performed, for example, by comparing to the past heart sound waveform data of the subject, or by comparing to the waveform data for the heart sound of a normal healthy person.

Consequently, the waveform including the first, second, third, and fourth sounds is analyzed from the heart sound data included in one heartbeat, and it can be determined whether there is any abnormality in the heart sound according to the difference from the past heart sound data for the subject for each of the sounds, the difference from the heart sound data for a typical healthy person, etc.

As a result, any abnormality in the heart sound can be detected more accurately than in the past.

The auscultation program according to the seventeenth invention is an auscultation program that measures the heart sound of a subject and determines whether there is any abnormality, the auscultation program causing a computer to execute an auscultation method comprising a data acquisition step, a data analysis step, and a determination step. In the data acquisition step, a data acquisition unit of an auscultation device acquires data about the heart sound. In the data analysis step, a data analysis unit of the auscultation device analyzes a waveform including a first sound, a second sound, a third sound, and a fourth sound included in the heart sound acquired in the data acquisition step. In the determination step, the determination unit of the auscultation device determines whether there is any abnormality in the heart sound on the basis of the analysis result for the waveform including the first sound, the second sound, the third sound, and the fourth sound analyzed in the data analysis step.

Here, the first sound, second sound, third sound, and fourth sound are heart sound sounds included in a single beat, and are made by the movement of the subject's heart (ventricle, atrium).

The first sound is, for example, a sound that occurs when a ventricle of the heart contracts. The second sound is, for example, a sound that occurs when a ventricle begins to expand. The third sound is, for example, a sound that occurs when a ventricle finishes expanding. The fourth sound is, for example, a sound that occurs when an atrium of the heart contracts.

Depending on the subject, all of the first, second, third, and fourth sounds may not be detected. For example, only the first and second sounds may be detected, or only the first, second, and third sounds may be detected, or only the first, second, and fourth sounds may be detected.

The determination of whether there is any abnormality in the heart sound by the determination unit is performed, for example, by comparing to the past heart sound waveform data of the subject, or by comparing to the waveform data for the heart sound of a normal healthy person.

Consequently, the waveform including the first, second, third, and fourth sounds is analyzed from the heart sound data included in one heartbeat, and it can be determined whether there is any abnormality in the heart sound according to the difference from the past heart sound data for the subject for each of the sounds, the difference from the heart sound data for a typical healthy person, etc.

As a result, abnormality in the heart sound can be detected more accurately than in the past.

### Effects

The auscultation device according to the present invention is able to detect abnormality in a heart sound more accurately than in the past.

### BRIEF DESCRIPTION OF THE DRAWINGS

FIG. 1 is a concept diagram showing the configuration of an auscultation system including a PC according to an embodiment of the present invention;

FIG. 2 is a control block diagram showing the configuration of the auscultation system in FIG. 1;

FIG. 3 is a graph comparing the waveform of a heart sound acquired by the communication unit of the PC in FIG. 2 to the waveform of an electrocardiogram;

FIG. 4 is a graph showing various parameters (signal strength (peak-to-peak), duration, waveform interval time, etc.) used for abnormality determination of the waveforms of sound I, sound II, sound III, and sound IV included in the heart sound waveform in FIG. 3;

FIG. 5 is a flowchart showing the flow of processing in an auscultation method executed by the PC in FIG. 2;

FIG. 6 is a control block diagram showing the configuration of an auscultation system including an auscultation device according to another embodiment of the present invention; and

FIG. 7 is a control block diagram showing the configuration of an auscultation system including an auscultation device according to yet another embodiment of the present invention.

### DETAILED DESCRIPTION OF THE EMBODIMENT

### Embodiment 1

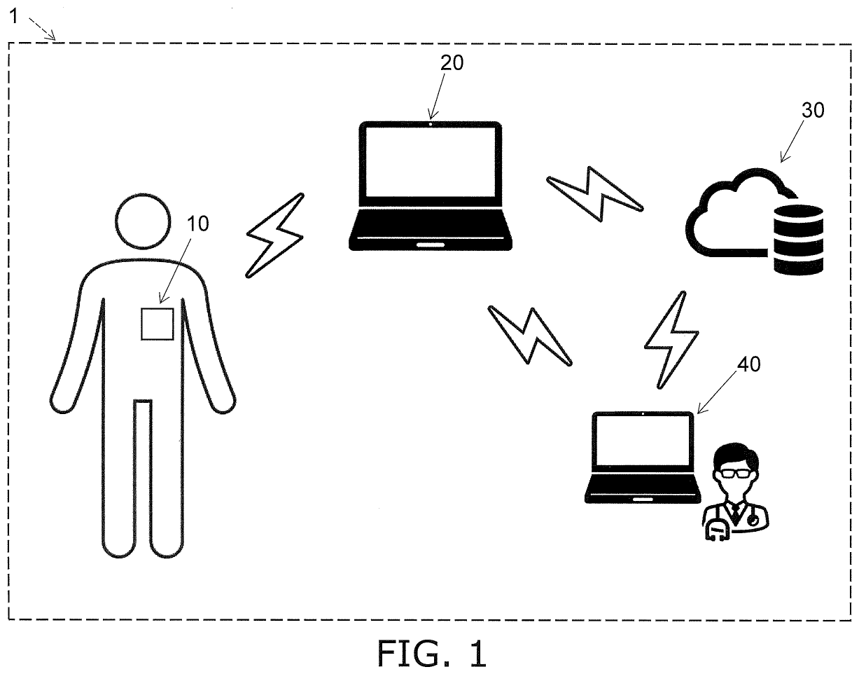

A PC (auscultation device) 20 according to an embodiment of the present invention and an auscultation system 1 comprising this PC will now be described using FIGS. 1 to 5.

In this embodiment, some unnecessarily detailed description may be omitted. For example, detailed description of already known facts or redundant description of components that are substantially the same may be omitted. This is to avoid unnecessary repetition in the following description, and facilitate an understanding on the part of a person skilled in the art.

The applicant has provided the appended drawings and the following description so that a person skilled in the art might fully understand this disclosure, but does not intend for these to limit what is discussed in the patent claims.

### (1) Configuration of Auscultation System 1

The auscultation system 1 according to this embodiment is a system that acquires and analyzes heart sound data for a subject measured with a stethoscope 10, and determines whether there is any abnormality in the heart sound, and comprises a stethoscope 10, a PC (personal computer) (auscultation device) 20, a cloud server 30, and a remote terminal 40, as shown in FIG. 1.

Here, the subject whose heart sound is measured with the stethoscope 10 may be, for example, a patient who has been hospitalized at medical facility, patient who is receiving home treatment, an elderly person, or a healthy person who has undergone health a checkup.

The stethoscope 10 is a device that measures the heart sound of a subject, and as shown in FIG. 1, for example, is attached to the chest of the subject, acquires data about the subject's heart sound, and transmits this to the PC 20 either wirelessly or over a wire.

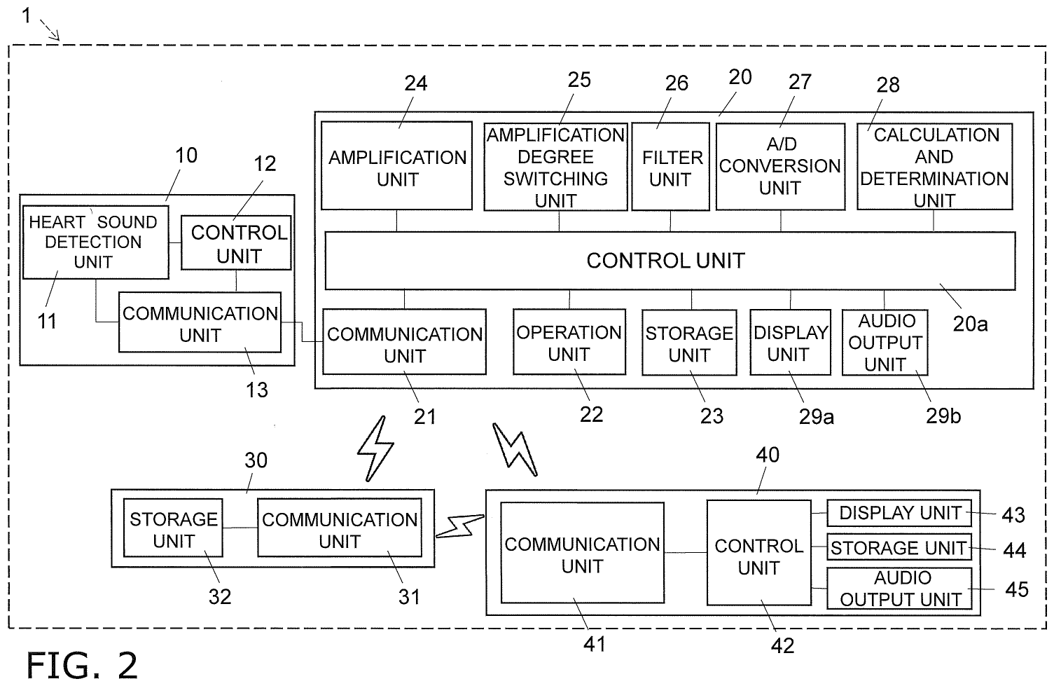

Also, the stethoscope 10 comprises a heart sound detection unit 11, a control unit 12, and a communication unit 13, as shown in FIG. 2.

The heart sound detection unit 11 is, for example, an acceleration sensor, a pressure sensor, a microphone, or the like, and detects a heart sound signal corresponding to the heart sound vibration of the subject as an electrical signal, and transmits this to the control unit 12.

The control unit 12 transmits an electrical signal indicating the heart sound detected by the heart sound detection unit 11 to the communication unit 13.

The communication unit 13 transmits the electrical signal indicating the heart sound received from the control unit 12 to the PC 20 as heart sound data. The transmission of data from the communication unit 13 to the PC 20 may be performed wirelessly or over a wire.

As shown in FIG. 1, the PC (auscultation device) 20 is able to communicate with the stethoscope 10, a cloud server 30, and a remote terminal 40, receives heart sound data measured by the stethoscope 10, and determines whether there is an abnormality in the heart sound of the subject.

The exact configuration of the PC 20 will be explained in detail below.

As shown in FIG. 1, the cloud server 30 is able to communicate with the PC 20 and the remote terminal 40, and mainly receives and stores heart sound data measured with the stethoscope 10, abnormality determination results for the PC 20, etc. Also, the cloud server 30 comprises a communication unit 31 and a storage unit 32, as shown in FIG. 2.

The communication unit 31 is wirelessly connected to the PC 20 and the remote terminal 40, and transmits and receives heart sound data, abnormality determination results, and so forth for each subject.

The storage unit 32 stores heart sound data, abnormality determination results, and the like received from the PC 20 via the communication unit 31.

The remote terminal 40 is, for example, a PC or other such terminal installed in a medical facility, etc., and as shown in FIG. 1, allows communication between the PC 20 and the cloud server 30, and mainly receives heart sound data measured with the stethoscope 10, the results of abnormality determination in the PC 20, etc., and displays these for a physician or the like.

Consequently, a physician at a medical facility can remotely check whether there is any abnormality in the heart sound of a patient or other such subject by displaying the heart sound data, abnormality determination results, etc., on the remote terminal 40.

Also, as shown in FIG. 2, the remote terminal 40 comprises a communication unit 41, a control unit 42, a display unit 43, a storage unit 44, and an audio output unit 45.

The communication unit 41 is wirelessly connected to the PC 20 and the cloud server 30, and transmits and receives heart sound data, abnormality determination results, etc., for each subject.

The control unit 42 controls the display unit 43 so as to display the heart sound data for the subject, the abnormality determination results, etc., received via the communication unit 41, and stores the data in the storage unit 44.

The display unit 43 is controlled by the control unit 42 and displays heart sound data, abnormality determination results, and so forth.

The storage unit 44 stores heart sound data, abnormality determination results, etc., received from the PC 20 via the communication unit 41.

The audio output unit 45 is controlled by the control unit 42 so as to reproduce the heart sound data acquired by the communication unit 41 on the basis of the determination result from the calculation and determination unit 28 on the PC 20 side (discussed below). This allows a physician or the like to listen to and check the heart sound at a remote site, according to the determination results.

With the auscultation system 1 of this embodiment, as discussed above, the PC 20 acquires and analyzes heart sound data for the subject measured with the stethoscope 10, determines whether there is any abnormality in the heart sound, and shares the heart sound data and abnormality determination results between the cloud server 30 and remote terminal 40.

Consequently, the heart sound measured with the stethoscope 10 is determined to be normal or abnormal, and if there is an abnormality or a possibility of an abnormality, a physician or the like can use the remote terminal 40 to check the data at any time, and not only in real-time diagnosis.

As a result, remote diagnosis can be performed, taking into account the risk of infection in a hospital during the coronavirus pandemic, the larger number of patients due to an aging society, the difficulty of ensuring transportation in rural areas, the societal pressures calling for telemedicine, and so forth, for example.

### (2) Configuration of PC 20

As shown in FIG. 2, the PC (auscultation device) 20 according to this embodiment comprises a control unit 20a, a communication unit (data acquisition unit) 21, an operation unit 22, a storage unit 23, an amplification unit 24, an amplification degree switching unit 25, a filter unit 26, an A/D conversion unit 27, a calculation and determination unit (data analysis unit, determination unit) 28, a display unit 29a, and an audio output unit (heart sound reproduction unit) 29b.

The control unit 20a is connected to the various units included in the PC 20 and controls those units.

The communication unit (data acquisition unit) 21 is able to communicate with the communication unit 13 of the stethoscope 10 either wirelessly or by wire, and as mentioned above, receives the heart sound data measured with the stethoscope 10, and stores this data in the storage unit 23 via the control unit 20a.

The operation unit 22 is, for example, an operation device such as a mouse or a keyboard that is connected to the PC 20, and receives the input of various operations.

The storage unit 23 is controlled by the control unit 20a so as to store the subject's heart sound data received via the communication unit 21. Also, the storage unit 23 stores the results of heart sound abnormality determination performed by the calculation and determination unit 28 (discussed below). Furthermore, the storage unit 23 stores information such as past heart sound data for each subject, determination results, heart sound data for typical healthy people, etc., used in determination processing in the calculation and determination unit 28 (discussed below).

The amplification unit 24 subjects the heart sound data received via the communication unit 21 to amplification processing and transmits the result to the control unit 20a.

The amplification degree switching unit 25 sets the amplification degree to what was inputted by the user via the operation unit 22.

The filter unit 26 performs filter processing on the heart sound data received via the communication unit 21 to remove noise and so on.

The A/D (analog/digital) conversion unit 27 is controlled by the control unit 20a so as to convert the heart sound data received via the communication unit 21 from an analog signal to a digital signal. The heart sound data converted into a digital signal is transmitted via the control unit 20a to the calculation and determination unit 28.

The calculation and determination unit (data analysis unit, determination unit) 28 uses the heart sound data that has undergone A/D conversion to analyze the waveform of the heart sound and determine whether there is any abnormality.

More specifically, the calculation and determination unit 28 analyzes the waveform including the sound I (first sound), sound II (second sound), sound III (third sound), and sound IV (fourth sound), and determines whether there is any abnormality in the heart sound on the basis of this analysis result.

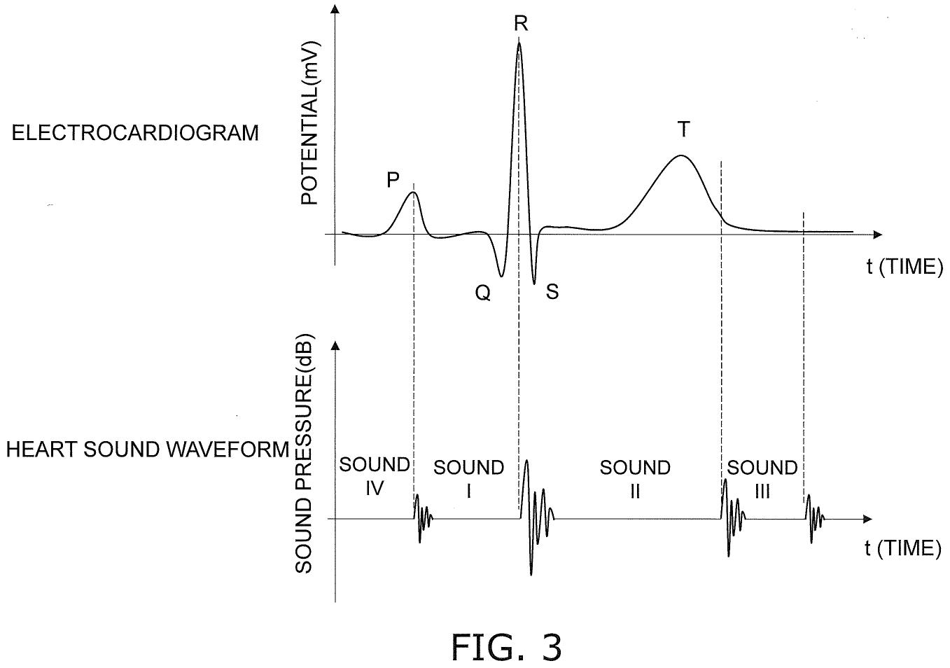

Here, as shown in FIG. 3, the data for one heartbeat of the heart sound includes the sound IV that appears at a point corresponding to the P wave that appears in the electrocardiogram waveform (electrical excitation of the atrium of the heart), the sound I that appears at a point corresponding to the R wave that appears in the electrocardiogram waveform (electrical excitation of the ventricle), the sound II that appears at a point corresponding to the T wave that appears in the electrocardiogram waveform (electrical recovery of ventricular excitation), and the sound III that appears at a point corresponding to the end of the T wave.

In FIG. 3, an electrocardiogram waveform is shown in the upper part in order to show the relation between the waveform of the heart sound detected by the heart sound detection unit 11 and the electrocardiogram waveform, but in this embodiment this does not mean that the waveform of the electrocardiogram is acquired with the stethoscope 10.

The sound I is a sound that occurs when a ventricle of the heart contracts. The sound II is a sound that occurs when ventricular expansion begins. The sound III is a sound that occurs when ventricular expansion has finished. The sound IV is a sound that occurs when an atrium of the heart contracts.

As shown in FIG. 3, the sound I and sound II tend to appear as larger waveforms than the sound III and sound IV, and depending on the subject, the sound III and sound IV may be extremely quiet.

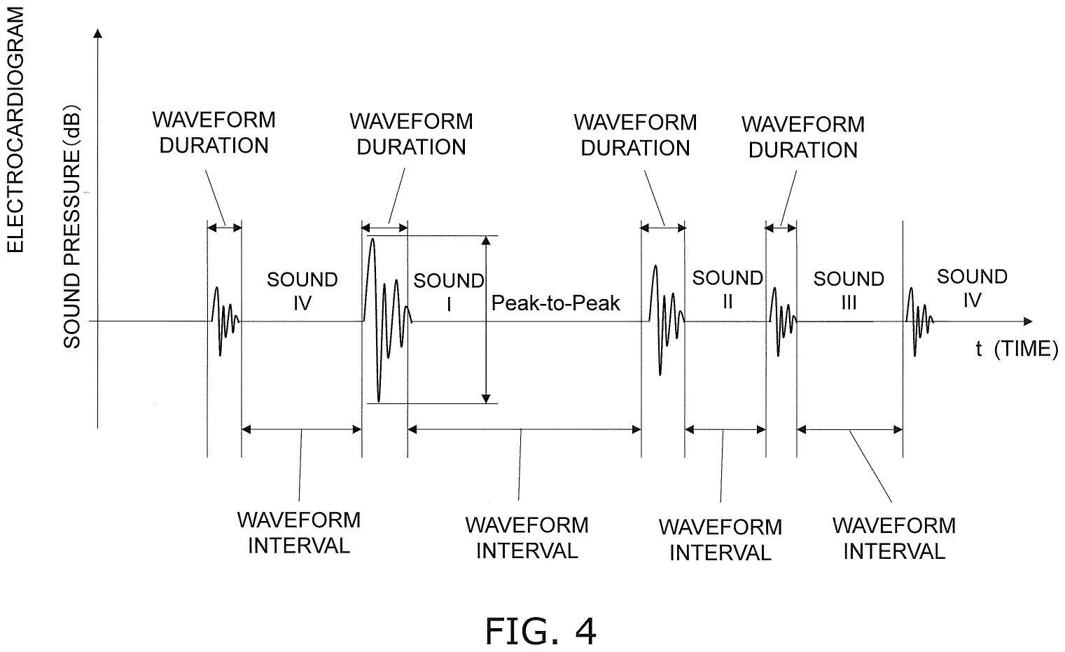

As shown in FIG. 4, the calculation and determination unit 28 determines whether there is any abnormality in the heart sound by using at least one of the signal strength (peak-to-peak), duration, and waveform interval of at least one waveform of at least one of the sound I (first sound), sound II (second sound), sound III (third sound), and sound IV (fourth sound) included in the data for a single heart sound.

The calculation and determination unit 28 compares the analyzed waveforms for the sound I, sound II, sound III, and sound IV with the waveforms for the sound I, sound II, sound III, and sound IV of the subject's past heart sounds, and determines whether there is any abnormality in the heart sound.

The calculation and determination unit 28 compares the analyzed waveforms for the sound I, sound II, sound III, and sound IV with the waveforms for the sound I, sound II, sound III, and sound IV of a typical healthy person, which serve as reference for determination, and determines whether there is any abnormality in the heart sound.

Furthermore, the calculation and determination unit 28 compares the analyzed waveforms for the sound I, sound II, sound III, and sound IV with the waveforms for the sound I, sound II, sound III, and sound IV of the subject's past heart sounds, calculates a difference value for a specific parameter (waveform signal strength, waveform duration, interval between waveforms, waveform characteristics, etc.), and determines whether there is any abnormality in the heart sound depending on whether the difference value is at or over a specific threshold.

Also, the calculation and determination unit 28 compares the analyzed waveforms for the sound I, sound II, sound III, and sound IV with the waveforms for the sound I, sound II, sound III, and sound IV of past heart sounds, and determines that the patient's condition requires follow-up observation in the event that the calculated difference value of the specific parameter is at or over a specific threshold, and the difference value of the specific parameter calculated by comparing to the waveform of a heart sound of a typical healthy person is below the specific threshold.

The calculation and determination unit 28 compares the analyzed waveforms for the sound I, sound II, sound III, and sound IV with the waveforms for the sound I, sound II, sound III, and sound IV of past heart sounds, and determines that the patient's condition requires follow-up observation in the event that the calculated difference value of the specific parameter is below a specific threshold, and the difference value of the specific parameter calculated by comparing to the waveforms of the heart sound of a typical healthy person is at or over the specific threshold.

The threshold used for determination using the difference value between the heart sound data measured with the stethoscope 10 and past heart sound data, and the threshold used for the determination using the difference value between the heart sound data measured with the stethoscope 10 and the heart sound data for typical healthy person are set for each parameter (waveform signal strength, waveform duration, interval between waveforms, waveform characteristics, etc.).

Also, the threshold used for determination using the difference value between heart sound data measured with the stethoscope 10 and past heart sound data, and the threshold used for determination using the difference value between the heart sound data measured with the stethoscope 10 and the heart sound data of a typical healthy person, may be the same values, or may be different values.

The display unit 29a is a liquid crystal display panel or the like of the PC 20, and is controlled by the control unit 20a so as to display the determination results from the calculation and determination unit 28, heart sound data waveforms, and so forth.

The audio output unit (heart sound reproduction unit) 29b is controlled by the control unit 20a so as to reproduce the heart sound data acquired by the communication unit 21 on the basis of the determination result from the calculation and determination unit 28.

### Auscultation Method

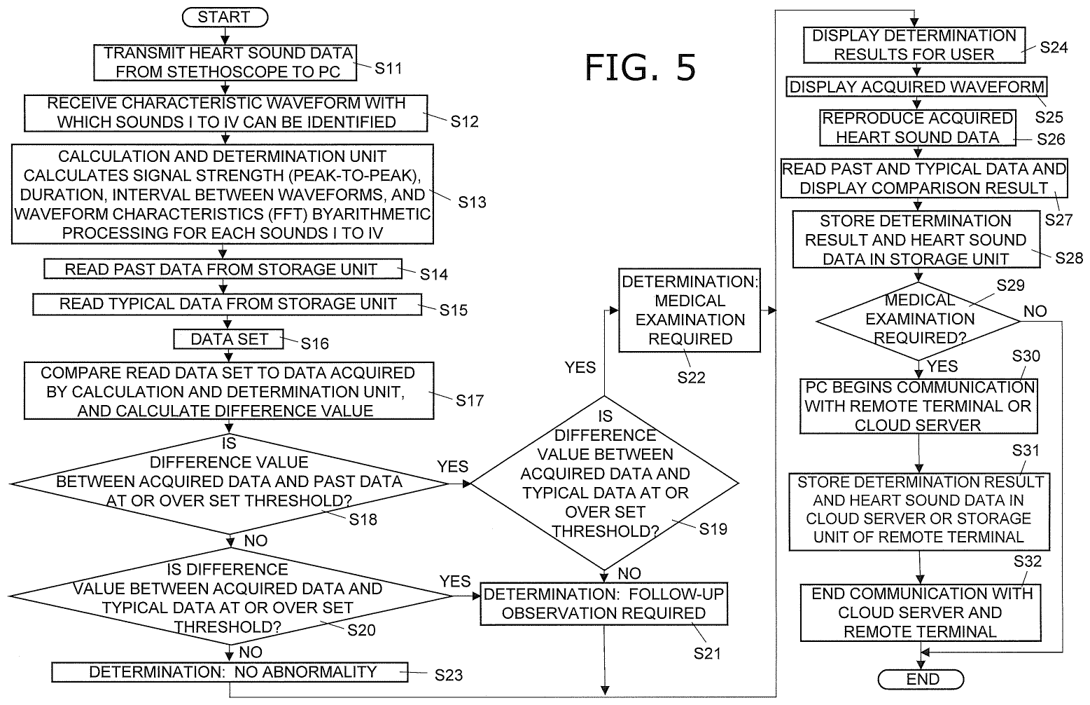

The PC 20 in this embodiment is configured as above and performs auscultation according to the method in the flowchart shown in FIG. 5.

That is, in step S11, the stethoscope 10 transmits the heart sound data detected by the heart sound detection unit 11 to the PC 20 via the communication unit 13.

Next, in step S12, the PC 20 receives, via the communication unit 21, the heart sound data (a waveform with which sounds I to IV can be identified) transmitted from the stethoscope 10.

Next, in step S13, the calculation and determination unit 28 of the PC 20 calculates the signal strength (peak-to-peak), duration, interval between waveforms, and waveform characteristics (FFT) by arithmetic processing.

Next, in step S14, the control unit 20a of the PC 20 reads, from the storage unit 23, heart sound data previously measured with the stethoscope 10 for the same subject.

Next, in step S15, the control unit 20a of the PC 20 reads, from the storage unit 23, heart sound data for a typical healthy person.

Next, in step S16, the calculation and determination unit 28 is set to compare the heart sound data measured with the stethoscope 10 and acquired by the PC 20, with the past heart sound data for the same subject read from the storage unit 23 by the control unit 20a and the heart sound data for a typical healthy person.

Next, in step S17, the calculation and determination unit 28 compares the heart sound data acquired by the PC 20 from the stethoscope 10 with past heart sound data for the same subject read from the storage unit 23 by the control unit 20a and the heart sound data for a typical healthy person, and calculates the difference value thereof.

Next, in step S18, it is determined whether the difference value between the heart sound data acquired by the PC 20 from the stethoscope 10 and the past heart sound data for the same subject read from the storage unit 23 by the control unit 20a is at or over a specific threshold.

Here, if the difference value is at or over the specific threshold, the processing proceeds to step S19, and if below the threshold, the processing proceeds to step S20.

Next, in step S19, since it was determined in step S18 that the difference value was at or over the specific threshold, the calculation and determination unit 28 determines whether the difference value between the heart sound data acquired from the stethoscope 10 by the PC 20 and the heart sound data for a typical healthy person read from the storage unit 23 by the control unit 20a is at or over a specific threshold.

Here, if the difference value is at or over the specific threshold, the processing proceeds to step S22, but if below the threshold, the processing proceeds to step S21.

Next, in step S20, since it was determined in step S18 that the difference value was below the specific threshold, the calculation and determination unit 28 determines whether the difference value between the heart sound data acquired from the stethoscope 10 by the PC 20 and the heart sound data of a typical healthy person read from the storage unit 23 by the control unit 20a is at or over a specific threshold.

Here, if the difference value is at or over the specific threshold, the processing proceeds to step S21, but if below the threshold, the processing proceeds to step S23.

Next, in step S21, since it was determined in step S18 that the difference value between the heart sound data acquired from the stethoscope 10 by the PC 20 and the past heart sound data for the same subject read from the storage unit 23 by the control unit 20a was below the specific threshold, and in step S20 that the difference value between the heart sound data acquired from the stethoscope 10 by the PC 20 and the heart sound data for a typical healthy person read from the storage unit 23 by the control unit 20a was at or over the specific threshold, the calculation and determination unit 28 determines that “follow-up observation is required.”

Similarly, in step S21, since it was determined in step S18 that the difference value between the heart sound data acquired by the PC 20 from the stethoscope 10 and the past heart sound data for the same subject read by the control unit 20a from the storage unit 23 was at or over a specific threshold, and in step S19 that the difference value between the heart sound data acquired from the stethoscope 10 by the PC 20 and the heart sound data for a typical healthy person read by the control unit 20a from the storage unit 23 was below the specific threshold, the calculation and determination unit 28 determines that “follow-up observation is required.”

Next, in step S22, since it was determined in step S18 that the difference value between the heart sound data acquired by the PC 20 from the stethoscope 10 and the past heart sound data for the same subject read by the control unit 20a from the storage unit 23 was at or over a specific threshold, and in step S19 that the difference value between the heart sound data acquired from the stethoscope 10 by the PC 20 and the heart sound data for a typical healthy person read by the control unit 20a from the storage unit 23 was at or over the specific threshold, the calculation and determination unit 28 determines that “medical examination is required.”

Next, in step S23, since it was determined in step S18 that the difference value between the heart sound data acquired by the PC 20 from the stethoscope 10 and the past heart sound data for the same subject read by the control unit 20a from the storage unit 23 was below a specific threshold, and in step S20 that the difference value between the heart sound data acquired from the stethoscope 10 by the PC 20 and the heart sound data for a typical healthy person read by the control unit 20a from the storage unit 23 was below the specific threshold, the calculation and determination unit 28 determines that there is “no abnormality”.

Next, in step S24, the control unit 20a of the PC 20 controls the display unit 29a so as to display the determination results in steps S21 to S23 (follow-up observation required, medical examination required, no abnormality).

Next, in step S25, the control unit 20a of the PC 20 controls the display unit 29a so as to display the waveform of the heart sound data measured and acquired with the stethoscope 10.

Next, in step S26, the control unit 20a of the PC 20 controls the audio output unit 29b so as to reproduce heart sound data corresponding to the determination result.

Next, in step S27, the control unit 20a of the PC 20 controls the display unit 29a so as to display the result of reading and comparing the past heart sound data for the same subject stored in the storage unit 23 and the heart sound data of a typical healthy person.

Next, in step S28, the control unit 20a of the PC 20 causes the storage unit 23 to store the determination result from the calculation and determination unit 28 and the heart sound data corresponding to this determination result.

Next, in step S29, it is determined whether or not the determination result was “medical examination required” (step S22) in steps S21 to S23.

Here, if the determination result was “medical examination required,” the processing proceeds to step S30, and if the determination result was anything other than “medical examination required,” the processing is ended at that point.

Next, in step S30, since it was determined in step S29 that the determination result in steps S21 to S23 was “medical examination required,” the PC 20 begins communication in order to transmit information such as heart sound data and determination results corresponding to “medical examination required” to the cloud server 30 and the remote terminal 40 via the communication unit 21 in order to provide information to the physician.

Next, in step S31, the cloud server 30 and the remote terminal 40 store the determination result and heart sound data received from the PC 20 in the storage unit 32 and the storage unit 44, respectively.

Next, in step S32, the PC 20 ends communication with the cloud server 30 and the remote terminal 40, and processing is terminated.

### Main Features

As shown in FIG. 2, the PC 20 in this embodiment comprises the communication unit 21 and the calculation and determination unit 28. The communication unit 21 acquires heart sound data from the stethoscope 10. The calculation and determination unit 28 analyzes the waveform including the sound I, sound II, sound III, and sound IV included in one heartbeat of the heart sound acquired by the communication unit 21. The calculation and determination unit 28 determines whether there is any abnormality in the heart sound on the basis of the analysis result for the analyzed waveform including the sound I, sound II, sound III, and sound IV.

Consequently, the waveform including the sound I, sound II, sound III, and sound IV is analyzed from the heart sound data included in one heartbeat, and whether there is any abnormality in the heart sound can be determined according to the difference for each sound from the past heart sound data for a subject, the difference from the heart sound data of a typical healthy person, etc.

As a result, whether there is any abnormality in the heart sound can be detected more accurately than in the past.

Also, the PC 20 acquires a waveform that has characteristics allowing the identification of the sound I, sound II, sound III, and sound IV included in the heart sound data, and can calculate, by arithmetic processing, the signal strength (peak-to-peak) for each waveform of the sounds I to IV, the waveform duration, the interval between waveforms, and waveform characteristics (FFT analysis, etc.).

Consequently, by comparing the obtained data with the subject's own data from the past or with data of a typical healthy person, a difference value between the past and typical data is calculated, and it is determined that there is no abnormality if the difference value is below a preset threshold, and that there is an abnormality if this value is at or over the threshold.

As a result, if it is determined that there is an abnormality, it is possible to take appropriate measures, such as informing the user through a display of the determination result, or transmitting the determination result to the remote terminal 40 used by a physician or the like.

### Embodiment 2

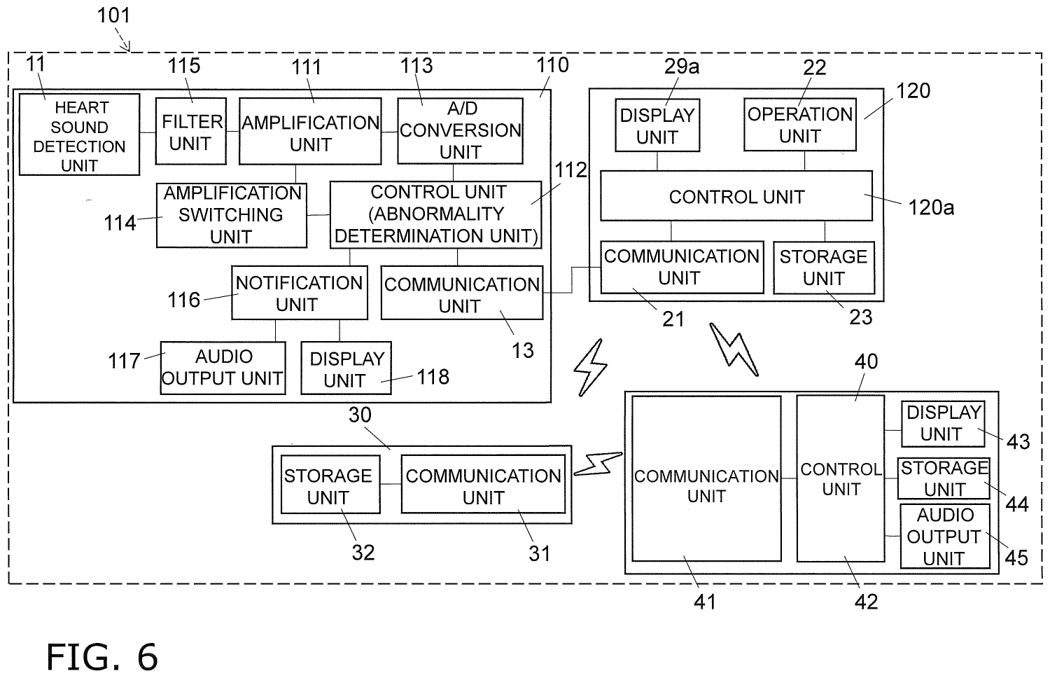

A stethoscope (auscultation device) 110 and an auscultation system 101 equipped with this stethoscope according to another embodiment of the present invention will now be described through reference to FIG. 6.

The auscultation system 101 of this embodiment differs from the configuration of Embodiment 1 above in that the main thing that performs abnormality determination using heart sound data is the stethoscope 110 rather than the PC 120.

The configuration other than the difference mentioned above is basically the same as that of Embodiment 1, so those components that appeared in Embodiment 1 above shall be numbered the same and not described again in detail.

That is, as shown in FIG. 6, the stethoscope 110 of this embodiment constitutes the auscultation system 101 together with a PC 120, the cloud server 30, and the remote terminal 40.

As shown in FIG. 6, the PC 120 comprises the communication unit 21, the operation unit 22, the storage unit 23, and a control unit 120a.

The control unit 120a controls the communication unit 21, the operation unit 22, the storage unit 23, and the display unit 29a.

As shown in FIG. 6, the stethoscope 110 comprises the heart sound detection unit (data measurement section) 11, an amplification unit 111, a control unit (data analysis unit, determination unit) 112, an A/D conversion unit 113, an amplification degree switching unit 114, a filter unit 115, a notification unit 116, a communication unit 13, an audio output unit (heart sound reproduction unit) 117, and a display unit 118.

The amplification unit 111 subjects the heart sound data detected by the heart sound detection unit 11 to amplification processing and transmits the result to the A/D conversion unit 113.

The control unit (data analysis unit, determination unit) 112 has a configuration corresponding to the calculation and determination unit 28 provided to the PC 20 of Embodiment 1, uses heart sound data that has undergone A/D conversion by the A/D conversion unit 113 to analyze the heart sound waveform, and determines whether there is any abnormality.

More specifically, the control unit 112 controls the sound I (first sound), sound II (second sound), sound III (third sound), and sound IV (fourth sound) included in data for one heartbeat of the heart sound, and determines whether there is any abnormality on the basis of this analysis result.

The A/D (analog-digital) conversion unit 113 is controlled by the control unit 112 so as to convert the heart sound data detected by the heart sound detection unit 11 from an analog signal to a digital signal.

The amplification degree switching unit 114 sets the amplification degree to what has been inputted by the user.

The filter unit 115 performs filter processing on the heart sound data detected by the heart sound detection unit 11 to remove noise and so on.

If a digital filter is used as the filter unit 115, it may be connected to the control unit 112. Also, if an analog filter is used as the filter unit 115, it may be inserted between the heart sound detection unit 11 and the amplification unit 111, as shown in FIG. 6.

The notification unit 116 is controlled by the control unit 112 so that notification of the determination result is performed on the audio output unit 117 and/or the display unit 118 depending on the result of the abnormality determination by the control unit 112.

The audio output unit (heart sound reproduction unit) 117 is controlled by the control unit 112 so as to reproduce the heart sound data detected by the heart sound detection unit 11 on the basis of the determination result at the control unit 112.

The display unit 118 is a liquid crystal display panel, etc., provided to the stethoscope 110, and is controlled by the control unit 112 so as to display the determination result at the control unit 112, the waveform of heart sound data, and so forth.

As discussed above, the stethoscope 110 in this embodiment determines whether there is any abnormality in the heart sound detected by the heart sound detection unit 11 within the stethoscope 110.

That is, the stethoscope 110 is a device that measures the heart sound of a subject and determines whether there is any abnormality, and comprises the heart sound detection unit (data measurement unit) 11 and the control unit 112. The heart sound detection unit 11 measures heart sound data. The control unit 112 analyzes a waveform including a first sound, second sound, third sound, and fourth sound included in one heartbeat of the heart sound measured by the heart sound detection unit 11. The control unit 112 determines whether there is any abnormality in the heart sound on the basis of the analysis result for the waveform including the analyzed first sound, second sound, third sound, and fourth sound.

Consequently, the waveform including the first, second, third, and fourth sounds can be analyzed from the heart sound data included in a single beat, and it can be determined whether there is any abnormality in the heart sound according to the difference from the past heart sound data of the subject, the heart sound data of a typical healthy person, etc.

As a result, whether there is any abnormality in the heart sound can be detected more accurately than in the past.

### Embodiment 3

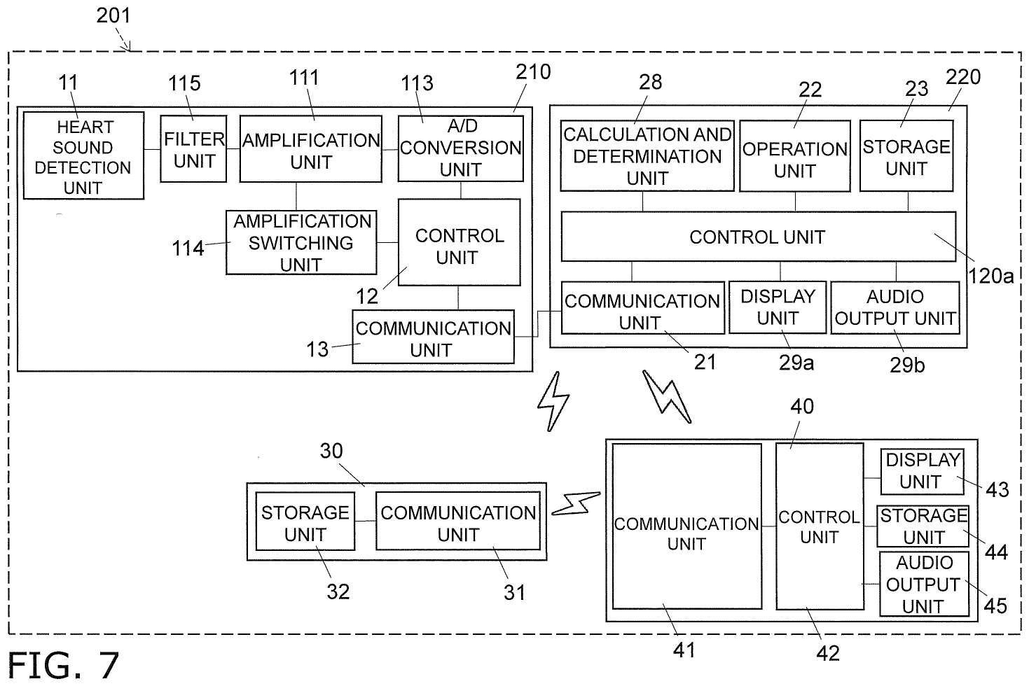

A stethoscope (auscultation device) 210, a PC (auscultation device) 220, and an auscultation system 201 including these according to yet another embodiment of the present invention will now be described through reference to FIG. 7.

The auscultation system 201 in this embodiment differs from the configurations of Embodiments 1 and 2 in that processing from detection of heart sound data to A/D conversion is performed on the stethoscope 210 side instead of at the PC 220, and the data that has undergone A/D conversion is sent to the PC 220 and the heart sound data is subjected to abnormality determination processing on the PC 220 side.

Other than the above difference, the configuration is basically the same as those of Embodiments 1 and 2, so those components that appeared in Embodiments 1 and 2 above shall be numbered the same and not described again in detail.

That is, as shown in FIG. 7, the stethoscope 210 in this embodiment constitutes the auscultation system 201 together with the PC 220, the cloud server 30, and the remote terminal 40.

As shown in FIG. 7, the stethoscope 210 comprises the heart sound detection unit (data measurement unit) 11, the filter unit 115, the amplification unit 111, the control unit 12, the A/D conversion unit 113, the amplification degree switching unit 114, and the communication unit 13.

Accordingly, with the stethoscope 210, the heart sound data detected by the heart sound detection unit 11 undergoes filter processing by the filter unit 115, undergoes amplification processing by the amplification unit 111, and is converted from analog data to digital data by the A/D conversion unit 113, after which the result is transmitted to the PC 220 via the communication unit 13.

Consequently, because the heart sound data detected by the heart sound detection unit 11 is transmitted to the PC 220 after having undergone amplification processing, there is less admixture of noise than with a configuration in which the heart sound data detected by the heart sound detection unit 11 is transmitted directly to the PC 220.

As shown in FIG. 7, the PC 220 comprises the communication unit 21, the operation unit 22, the storage unit 23, the calculation and determination unit 28, the display unit 29a, the audio output unit 29b, and the control unit 120a.

The control unit 120a controls the communication unit 21, the operation unit 22, the storage unit 23, the calculation and determination unit 28, the display unit 29a, and the audio output unit 29b.

The calculation and determination unit 28 acquires, via the communication unit 21, heart sound data that has undergone amplification processing and A/D conversion processing on the stethoscope 210 side, and uses heart sound data with relatively little noise to determine whether there is any abnormality.

Consequently, just as in Embodiments 1 and 2 above, the waveform including the first, second, third, and fourth sounds is analyzed from the heart sound data included in one heartbeat, and whether there is any abnormality in the heart sound can be determined according to the difference from past heart sound data for the subject, the difference from the heart sound data of a typical healthy person, and so forth.

As a result, abnormality in the heart sound can be detected more accurately than in the past.

### OTHER EMBODIMENTS

Embodiments of the present invention were described above, but the present invention is not limited to or by the above embodiments, and various changes are possible without departing from the gist of the invention.

### (A)

In the embodiments described above, an example was given in which the present invention was realized as the stethoscope 10 and an auscultation method. However, the present invention is not limited to this.

For example, the present invention may be realized as an auscultation program that causes a computer to execute the above-mentioned auscultation method.

This auscultation program is stored in a memory (storage unit) installed in an auscultation device, and a CPU reads the auscultation program stored in the memory and causes the hardware to execute the steps. More specifically, the same effects as described above can be obtained when a CPU reads an auscultation program and executes the above-mentioned data acquisition step, data analysis step, and determination step.

Also, the present invention may be realized as a recording medium that stores an auscultation program.

### (B)

In the embodiments described above, an example was given in which the calculation and determination unit 28 of the PC 20 or the control unit 112 of the stethoscope 110 compared past heart sound data for a given subject and heart sound data of a typical healthy person to heart sound data measured with the stethoscopes 10 or 110, and it was determined whether there was any abnormality. However, the present invention is not limited to this.

For example, what is to be compared with heart sound data measured with a stethoscope may be just the past data for a given subject or the heart sound data of a typical healthy person.

Here again, it is possible to determine whether or not there is any abnormality in the heart sound of the subject.

### (C)

In the embodiments described above, an example was given in which the determination result related to the heart sound data measured with the stethoscope 10 was determined in three stages: medical examination required, follow-up observation required, and no abnormality. However, the present invention is not limited to this.

For example, thresholds for determination may be set in multiple stages, and in addition to the three-stage determination results of medical examination required, follow-up observation required, and no abnormality, determination may be made more precisely in four or more stages.

### (D)

In the embodiments described above, an example was given in which the signal strength and duration of the waveform of sound I, sound II, sound III, and sound IV included in the heart sound waveform, the waveform interval, and so forth were used as shown in FIG. 4 as the parameter used to determine there was any abnormality in the heart sound. However, the present invention is not limited to this.

For example, characteristics of the waveform of sound I, sound II, sound III, and sound IV included in the heart sound waveform (such as the frequency characteristics based on FFT (fast Fourier transform) analysis, etc.) may be used as parameters for abnormality determination.

### (E)

In Embodiment 1, an example was given in which the PC 20 was used as an auscultation device that received heart sound data from the stethoscope 10 and determined whether there was any abnormality. However, the present invention is not limited to this.

For example, instead of a PC, a device such as a smartphone or a tablet terminal may be used as the auscultation device.

### (F)

In the embodiments described above, an example was given in which it was determined whether there was any abnormality in the heart sound of a subject. However, the present invention is not limited to this.

For example, whether there is any abnormality in a subject is not limited to heart sound, and may be determined using waveforms of other body sounds, such as breath sounds, lung sounds, and bowel sounds.

### (G)

In the above embodiment, an example was given in which the stethoscope 10 was used in a state of being attached to the chest of the subject. However, the present invention is not limited to this.

For example, the present invention may be applied to a digital stethoscope with which a physician or the like places an auscultation part on the chest, etc., of the subject to acquire heart sound data.

### Addenda

The auscultation device according to the fourth invention may be the auscultation device according to any of the first to third inventions, wherein the determination unit determines whether there is any abnormality in the heart sound by comparing the waveform analyzed by the data analysis unit to a past heart sound waveform for the subject.

The auscultation device according to the fifth invention may be the auscultation device according to any of the first to fourth inventions, wherein the determination unit determines whether there is any abnormality in the heart sound by comparing the waveform analyzed by the data analysis unit to the waveform of the heart sound of a typical healthy person.

The auscultation device according to the ninth invention may be the auscultation device according to any of the first to eighth inventions, wherein the first sound is a sound that occurs when a ventricle of the heart contracts, and the second sound is a sound that occurs when the ventricle begins to expand.

The auscultation device according to the tenth invention may be the auscultation device according to any of the first to ninth inventions, wherein the third sound is a sound that occurs when the ventricle finishes expanding, and the fourth sound is a sound that occurs when an atrium of the heart contracts.

The auscultation device according to the eleventh invention may be the auscultation device according to any of the first to tenth inventions, further comprising a display unit that displays the determination result at the determination unit.

The auscultation device according to the twelfth invention may be the auscultation device according to any of the first to eleventh inventions, further comprising a heart sound reproduction unit that reproduces heart sound data acquired by the data acquisition unit on the basis of the determination result at the determination unit.

The auscultation device according to the thirteenth invention may be the auscultation device according to any of the first to twelfth inventions, further comprising a storage unit that stores the heart sound data acquired at the data acquisition unit and the determination result at the determination unit.

The auscultation system according to the fourteenth invention comprises the auscultation device according to any of the first to thirteenth inventions; and a remote terminal to which the heart sound data and the determination result are transmitted from the auscultation device.

### INDUSTRIAL APPLICABILITY

The auscultation device of the present invention exhibits the effect that whether there is any abnormality in a heart sound can be detected more accurately than in the past, and therefore can be broadly applied to various devices that measure and analyze heart sound data.

### REFERENCE SIGNS LIST

1 auscultation system
10 stethoscope
11 heart sound detection unit
12 control unit
13 communication unit
20 PC (auscultation device)
20a control unit
21 communication unit (data acquisition unit)
22 operation unit
23 storage unit
24 amplification unit
25 amplification degree switching unit
26 filter unit
27 A/D conversion unit
28 calculation and determination unit (data analysis unit, determination unit)
29a display unit
29b audio output unit (heart sound reproduction unit)
30 cloud server
31 communication unit
32 storage unit
40 remote terminal
41 communication unit
42 control unit
43 display unit
44 storage unit
101 auscultation system
110 stethoscope (auscultation device)
111 amplification unit
112 control unit (data analysis unit, determination unit)
113 A/D conversion unit
114 amplification switching unit
115 filter unit
116 notification unit
117 audio output unit (heart sound reproduction unit)
118 display unit
120 PC
120a control unit
201 auscultation system
210 stethoscope
220 PC (auscultation device)

What is claimed is:

1. An auscultation device that acquires heart sound data from a stethoscope that measures a heart sound of a subject, and determines whether there is any abnormality, the auscultation device comprising:
a data acquisition unit configured to acquire the heart sound data from the stethoscope;
a data analysis unit configured to analyze a waveform including a first sound, a second sound, a third sound, and a fourth sound included in one heartbeat of the heart sound acquired by the data acquisition unit; and
a determination unit configured to determine whether there is any abnormality in the heart sound on the basis of an analysis result for the waveform including the first sound, the second sound, the third sound, and the fourth sound analyzed by the data analysis unit.

2. An auscultation device that measures a heart sound of a subject and determines whether there is any abnormality, the auscultation device comprising:
a data measurement unit configured to measure a heart sound data;
a data analysis unit configured to analyze a waveform including a first sound, a second sound, a third sound, and a fourth sound included in one heartbeat of the heart sound measured by the data measurement unit; and
a determination unit configured to determine whether there is any abnormality in the heart sound on the basis of an analysis result for the waveform including the first sound, the second sound, the third sound, and the fourth sound analyzed by the data analysis unit.

3. The auscultation device according to claim 1,
wherein the determination unit determines whether there is any abnormality in the heart sound by using the analysis result by the data analysis unit, a signal strength and duration of at least one of the first sound, the second sound, the third sound, and the fourth sound, an interval between the waveforms, and at least one of the waveform characteristics.

4. The auscultation device according to claim 1,
wherein the determination unit determines whether there is any abnormality in the heart sound by comparing the waveform analyzed by the data analysis unit to a waveform of a past heart sound of the subject.

5. The auscultation device according to claim 4,
wherein the determination unit determines whether there is any abnormality in the heart sound by comparing the waveform analyzed by the data analysis unit to the waveform of the heart sound of a typical healthy person.

6. The auscultation device according to claim 4,
wherein the determination unit compares the waveform of the heart sound analyzed by the data analysis unit to a past heart sound waveform, calculates a difference value of a specific parameter, and determines whether there is any abnormality in the heart sound depending on whether the difference value is at or over a specific threshold.

7. The auscultation device according to claim 5,
wherein the determination unit determines that a condition of the subject requires follow-up observation in an event that a difference value of a specific parameter calculated by comparing the waveform of the heart sound analyzed by the data analysis unit to a past heart sound waveform is at or over a specific threshold, and the difference value of a specific parameter calculated by comparing to the waveform of the heart sound of a typical healthy person is below a specific threshold.

8. The auscultation device according to claim 5,
wherein the determination unit determines that a condition of the subject requires follow-up observation in an event that a difference value of a specific parameter calculated by comparing the waveform of the heart sound analyzed by the data analysis unit to a past heart sound waveform is below a specific threshold, and the difference value of a specific parameter calculated by comparing to the waveform of the heart sound of a typical healthy person is at or over a specific threshold.

9. The auscultation device according to claim 1,
wherein the determination unit determines whether there is any abnormality in the heart sound by comparing the waveform analyzed by the data analysis unit to the waveform of the heart sound of a typical healthy person.

10. The auscultation device according to claim 1,
wherein the first sound is a sound that occurs when a ventricle of heart contracts, and the second sound is the sound that occurs when a ventricle begins to expand.

11. The auscultation device according to claim 10,
wherein the third sound is the sound that occurs when a ventricle finishes expanding, and the fourth sound is the sound that occurs when an atrium of the heart contracts.

12. The auscultation device according to claim 1,
further comprising a display unit configured to display a determination result from the determination unit.

13. The auscultation device according to claim 1,
further comprising a heart sound reproduction unit configured to reproduce data about the heart sound on the basis of a determination result from the determination unit.

14. The auscultation device according to claim 1,
further comprising a storage unit configured to store the heart sound data and a determination result from the determination unit.

15. An auscultation system, comprising:
the auscultation device according to claim 1; and
a remote terminal to which the heart sound data and a determination results are transmitted from the auscultation device.

16. An auscultation method for measuring a heart sound of a subject and determining whether there is any abnormality, the method comprising:
a data acquisition step in which a data acquisition unit of an auscultation device acquires data about the heart sound;
a data analysis step in which a data analysis unit of the auscultation device analyzes a waveform including a first sound, a second sound, a third sound, and a fourth sound included in the heart sound acquired in the data acquisition step; and
a determination step in which a determination unit of the auscultation device determines whether there is any abnormality in the heart sound on the basis of an analysis result for the waveform including the first sound, the second sound, the third sound, and the fourth sound analyzed in the data analysis step.

17. An auscultation program that measures a heart sound of a subject and determines whether there is any abnormality, the auscultation program causing a computer to execute an auscultation method comprising:
a data acquisition step in which a data acquisition unit of an auscultation device acquires data about the heart sound;
a data analysis step in which a data analysis unit of the auscultation device analyzes a waveform including a first sound, a second sound, a third sound, and a fourth sound included in the heart sound acquired in the data acquisition step; and
a determination step in which a determination unit of the auscultation device determines whether there is any abnormality in the heart sound on the basis of an analysis result for the waveform including the first sound, the second sound, the third sound, and the fourth sound analyzed in the data analysis step.
